(19) United States
(12) Patent Application Publication
(10) Pub. No.: US 2016/0039890 A1
(43) Pub. Date: Feb. 11, 2016

(54) INTRAOCULAR ANGIOGENESIS INHIBITOR AND USES THEREOF

(71) Applicant: Hideaki Hara, Gifu-shi (JP)

(72) Inventors: Hideaki Hara, Gifu-shi (JP); Yuki Inoue, Gifu-shi (JP)

(21) Appl. No.: 14/622,035

(22) Filed: Feb. 13, 2015

(30) Foreign Application Priority Data
Aug. 6, 2014 (JP) 2014-160077

(51) Int. Cl. C07K 14/34; A61K 38/16

(52) U.S. Cl. CPC C07K 14/34; A61K 38/164

(57) ABSTRACT

Intended is to provide a new therapeutic or prophylactic means for intraocular angiogenesis. Provided is an intraocular angiogenesis inhibitor including a polypeptide which is a variant of diphtheria toxin, and shows activity inhibiting binding between HB-EGF and an EGF receptor.

### CROSS REFERENCE TO RELATED APPLICATIONS

This application claims priority to Japanese application No. 2014-160077, filed Aug. 6, 2014, which is hereby incorporated by reference in its entirety.

### FIELD OF THE INVENTION

The present invention relates to an intraocular angiogenesis inhibitor, and more specifically to an intraocular angiogenesis inhibitor targeting a heparin-binding EGF-like growth factor (HB-EGF), and a prophylactic or therapeutic medicine for ophthalmologic diseases using it.

### BACKGROUND OF THE INVENTION

At present, the first cause of blindness due to sight disorder in Japan is glaucoma, and the total of diabetic retinopathy and age-related macular degeneration developed by abnormal angiogenesis accounts the largest percent (28.1%). Thus, the major cause of marked sight disorder in Japan and developed countries is regarded intraocular angiogenesis diseases, and typical examples include diabetic retinopathy, retinopathy of prematurity, age-related macular degeneration, occlusion of retinal vein, and neovascular glaucoma. The intraocular angiogenesis causes abnormal newborn blood vessels from the existing vessels in the retina, chorioid, and iris.

The mechanism of intraocular angiogenesis is said to be closely related with vascular endothelium growth factor (VEGF). VEGF is an important factor acting specifically on vascular endothelial cells during blood vessel formation. On the cell level, VEGF accelerates proliferation and inhibits apoptosis of endothelial cells, and on the individual level, VEGF induces angiogenesis, increased vascular permeability, migration of vascular endothelial cells, lumen formation, production of clotting and fibrinolytic proteins from endothelial cells, and expression of cell adhesion molecules on endothelial cells. At present, three therapeutic agents, Pegaptanib (Macugen (registered trademark)), Ranibizumab (Lucentis (registered trademark)), and Aflibercept (VEGF Trap-eye (registered trademark)) are used in Japan. Pegaptanib is the first approved anti-VEGF drug (nucleic acid preparation) in the ophthalmological field in the world, and has high safety. However, it can only block VEGF₁₆₅, so that its anti-angiogenic action is limited. On the other hand, Ranibizumab is a Fab fragment of human anti-VEGF monoclonal antibody which inhibits all isoforms of VEGF-A, and has strong anti-VEGF action. Furthermore, Aflibercept bounds as a soluble decoy acceptor with VEGF and a placenta growth factor with a higher affinity than a natural acceptor, and thereby inhibiting binding with the intrinsic VEGF acceptor and its activation. A certain effect is found in clinical cases, but there are problems that complete recovery cannot be achieved only by them, and that antibody therapy imposes a heavy burden on the body because of its intravitreal administration.

### SUMMARY OF THE INVENTION

The present invention is intended to provide a new prophylactic and therapeutic means for intraocular angiogenesis.

During the investigation in consideration of the above-described problems, the inventors focused on the heparin-binding epidermal growth factor-like growth factor (HB-EGF). HB-EGF is one of the EGF family, and is reported to be involved in carcinogenesis and angiogenesis of cells. HB-EGF is also known to be expressed intraocularly. However, the action of HB-EGF on intraocular angiogenesis has not been clarified. Therefore, various experiments were carried out for clarifying the role of HB-EGF in intraocular angiogenesis. As a result of this, it was found that HB-EGF is closely involved with intraocular angiogenesis, and is a potential therapeutic target. As a result of further study, the action of HB-EGF (cell proliferation and promotion of migration) are effectively suppressed by CRM-197, which is an HB-EGF inhibitor. CRM-197 is a variant of diphtheria toxin, and shows activity of inhibiting binding between HB-EGF and an EGF receptor (for example, see JP2004-155776 and WO2006/137398). In recent investigation, involvement of HB-EGF in cancer angiogenesis is reported, and CRM-197 is in the clinical trial phase II targeting ovarian cancer.

The invention described below is based on the above-described results and observations.

[1] A prophylactic or therapeutic method for an ophthalmologic disease accompanied by intraocular angiogenesis, including a step of administering a polypeptide to a patient with ophthalmologic disease accompanied by intraocular angiogenesis in a therapeutically effective amount, wherein the polypeptide is a variant of diphtheria toxin and shows an activity of inhibiting binding between HB-EGF and an EGF receptor.

[2] The method of [1], wherein the polypeptide is CRM-197.

[3] The method of [1], wherein the ophthalmologic disease is age-related macular degeneration, diabetic retinopathy, neovascular glaucoma, proliferative diabetic retinopathy, retinopathy of prematurity, exudative age-related macular degeneration, neovascular glaucoma, occlusion of retinal vein, retinal artery obstruction, pterygium, rubeosis, or corneal neovascularization.

[4] The method of [1], wherein the ophthalmologic disease is diabetic retinopathy, proliferative diabetic retinopathy, or exudative age-related macular degeneration.

[5] An intraocular angiogenesis inhibitor including a polypeptide which is a variant of diphtheria toxin, and shows activity inhibiting binding between HB-EGF and an EGF receptor.

[6] The intraocular angiogenesis inhibitor of [5], wherein the polypeptide is CRM-197.

[7] A prophylactic or therapeutic medicine for an ophthalmologic disease accompanied by intraocular angiogenesis, including the intraocular angiogenesis inhibitor of [5] as an active ingredient.

[8] The prophylactic or therapeutic medicine of [7], wherein the ophthalmologic disease is age-related macular degeneration, diabetic retinopathy, neovascular glaucoma, proliferative diabetic retinopathy, retinopathy of prematurity, exudative age-related macular degeneration, neovascular glaucoma, occlusion of retinal vein, retinal artery obstruction, pterygium, rubeosis, or corneal neovascularization.

The intraocular angiogenesis inhibitor of the present invention provides a new treatment strategy for ophthalmologic diseases accompanied by intraocular angiogenesis. Newly formed blood vessels are vulnerable and easily broken in comparison with normal blood vessels, so that cause leakage of blood components from blood vessels and bleeding to damage the retina, and cause visual decrease. Human obtains about 80% of information by eyes, so that visual decrease is burden on daily life of the patient, and can deteriorate the quality of life (QOL). Under these circumstances, the present invention introduces a novel treatment means, and plays a significant role. The present invention is also effective as a novel treatment approach for patients who cannot be treated with anti-VEGF therapy alone. The intravitreal administration in anti-VEGF therapy imposes a heavy burden on the patient. Therefore, the use of the present invention for the patient to whom anti-VEGF therapy is effective decreases the number of intravitreal administration and improves the QOL of the patient. Accordingly, the present invention is useful also for the patient to whom anti-VEGF therapy is effective.

### BRIEF DESCRIPTION OF THE DRAWINGS

These and other objectives and technical advantages of the present invention will be readily apparent from the following description of the preferred exemplary embodiments of the invention in conjunction with the accompanying drawings, in which:

FIG. 1 Wound healing assay

FIG. 2 Role of HB-EGF on mouse CNV formation. Laser spots were photographed just after laser treatment and fluorescein angiogram images of laser-induced CNV were photographed at 2 weeks after laser treatment. (A) Representative CNV lesions of the choroidal flat mounts are shown. Scale bar; 100 μm. (B) Quantification of the size of CNV area. Data are shown as mean±S.E.M (n=9 or 10). *, p<0.05, vs. WT. (Student's t-test).

FIG. 3 Role of HB-EGF on mouse Oxygen-induced retinopathy model. Shown are original images and the analysis images obtained using the angiogenesis tube formation module in Metamorph. Scale bar; 500 or 250 μm (A). HB-EGF KO mice reduced bith the number of nodes and the node area. Data are shown as mean±S.E.M (n=6). *, p<0.05, **, p<0.01 vs. WT. (Student's t-test).

FIG. 4 Expression of HB-EGF and VEGF after laser-irradiation in RPE-choroidal. Representative band images show immunoreactivities against HB-EGF (A) and VEGF (C). HB-EGF was significantly increased at 3 days after laser induced CNV (B). VEGF was significantly increased at 5 and 7 days after laser induced CNV (D). Data are shown as mean±S.E.M (n=4 to 6). *, p<0.05, **, p<0.01 vs. Normal group (Dunnett's test).

The expression of HB-EGF and VEGF were merged after laser irradiation in neovascular reagion (E).

FIG. 5 HB-EGF-induced proliferation in HRMECs. HRMECs were cultured in a 96-well plate (at a density of 2,000 cells/well), and incubated for 24 h and starved for 24 h at 37° C. in 5% CO2. The cells were then supplemented with the indicated concentrations of HB-EGF, VEGF or HB-EGF and VEGF for 48 h, and measurements were made by WST-8 assay. HB-EGF was increased cell proliferation. Moreover, Both VEGF and HB-EGF treatment were increased cell proliferation more than VEGF-treated group. Data are shown as mean±S.E.M. (n=6). *, p<0.05, **, p<0.01 vs. Control, #, p<0.05; ##, p<0.01 vs. VEGF treated group (Dunnett's test).

FIG. 6 HB-EGF-induced migration of HRMEC in an in vitro wound-healing assay. Migration of HRMEC was assessed using a wound-healing assay. Briefly, 90% confluent monolayers of HRMEC were scratch-wounded, and then incubated for 24 h. Images of the wounded monolayer of HRMEC were taken at times 0 and 24 h after treatment for 24 h with HB-EGF or VEGF. The horizontal lines indicate the wound edge. Wound closure was increased compared to the controls by addition of HB-EGF, VEGF, or HB-EGF and VEGF. Migration was estimated by measurement of cell numbers within the wounded region. Data are shown as mean±S.E.M. (n=4). **, p<0.01 vs. Control, ##, p<0.01 vs. VEGF treated group (Dunnett's test).

FIG. 7 Effect of CRM-197 against HB-EGF-induced proliferation in HRMECs. HRMEC were cultured in a 96-well plate (at a density of 2,000 cells/well), and incubated for 24 h and starved for 24 h at 37° C. in 5% CO₂. The cells were then supplemented with the indicated concentrations of HB-EGF, VEGF or HB-EGF and VEGF for 48 h, and measurements were made by WST-8 assay. CRM-197 was added 1 h before treatment with HB-EGF, VEGF or HB-EGF and VEGF. CRM-197 was decreased HB-EGF, VEGF or HB-EGF and VEGF-induced cell proliferation. Data are shown as mean±S.E.M (n=5 or 6). ** p<0.01, vs. Control, ## p<0.01, vs. VEGF. $$ p<0.01 vs. HB-EGF, ‡‡p<0.01 vs. HB-EGF+VEGF (Tukey's test).

FIG. 8 Effect of CRM-197 against HB-EGF induced wound-healing assay in HRMECs. Migration of HRMEC was assessed using a wound-healing assay. Briefly, 90% confluent monolayers of HRMEC were scratch-wounded, and then incubated for 24 h. Images of the wounded monolayer of HRMEC were taken at times 0 and 24 h after treatment for HB-EGF or VEGF. The horizontal lines indicate the wound edge. CRM-197 was added 1 h before treatment with HB-EGF or VEGF. Wound closure was decreased compared to the representative controls by addition of CRM-197. Migration was estimated by measurement of cell numbers within the wounded region. Data are shown as mean±S.E.M (n=4). ** p<0.01, vs. Control, # p<0.05, ## p<0.01, vs. VEGF. $$ p<0.01 vs. HB-EGF, ‡‡ p<0.01 vs. HB-EGF+VEGF. (Tukey's test).

### DETAILED DESCRIPTION OF THE INVENTION

The present invention relates to an intraocular angiogenesis inhibitor. The polypeptide (more specifically, the active ingredient) composing the intraocular angiogenesis inhibitor of the present invention is a variant of diphtheria toxin, and has activity of inhibiting binding between HB-EGF and EGF receptor. Examples of the applicable polypeptide include CRM-197 and DT52E148. Of these examples, CRM-197 is preferred. For details about the active ingredient of the present invention, see the above-listed Patent Literatures (JP2004-155776 and WO2006/137398).

The intraocular angiogenesis inhibitor of the present invention is typically used for prevention or treatment of ophthalmologic diseases accompanied by intraocular angiogenesis. Accordingly, the present invention provides a prophylactic or therapeutic medicine for ophthalmologic diseases accompanied by intraocular angiogenesis (hereinafter collectively referred to as “the medicine of the present invention”), the medicine including the above-described intraocular angiogenesis inhibitor as an active ingredient.

The medicine of the present invention may be prepared into, for example, an injection for intravitreal injection, an instillation, or an eye ointment according to a common procedure. The injection for intravitreal injection can be produced by, for example, dissolving the above-described active ingredient in an appropriate solvent (for example, distilled water or a normal saline solution for injection). As needed, an isotonizing agent such as mannitol, sodium chloride, glucose, sorbit, glycerol, xylitol, fructose, maltose, or mannose, a stabilizer such as albumin, and a preservative such as benzyl alcohol, or methyl parahydroxybenzoate may be added into the preparation. In addition, an acid such as citric acid, or a base such as diisopropanolamine may be added into the preparation as a pH controlling agent. The injection for intravitreal injection may be a freeze dry preparation to be dissolved before use.

The instillation can be produced by, for example, dissolving the above-described active ingredient in an appropriate solvent (for example, distilled water or a normal saline solution for injection). As needed, an isotonizing agent such as mannitol, sodium chloride, glucose, sorbit, glycerol, xylitol, fructose, maltose, mannose, or glycerin, a stabilizer such as sodium edentate or albumin, a preservative such as benzyl alcohol or methyl parahydroxybenzoate, and a surface active agent such as polyoxyethylene monooleate or polyoxyl stearate 40 may be added into the preparation. In addition, an acid such as citric acid, or a base such as diisopropanolamine may be added into the preparation as a pH controlling agent.

The medicine of the present invention may be a mix agent including other prophylactic or therapeutic medicine used for ophthalmologic diseases accompanied by angiogenesis.

Examples of the ophthalmologic disease accompanied by angiogenesis to be prevented or treated by the medicine of the present invention include age-related macular degeneration, diabetic retinopathy, neovascular glaucoma, proliferative diabetic retinopathy, retinopathy of prematurity, exudative age-related macular degeneration, neovascular glaucoma, occlusion of retinal vein, retinal artery obstruction, pterygium, rubeosis, and corneal neovascularization. Among them, the medicine is particularly effective for diabetic retinopathy, proliferative diabetic retinopathy, and exudative age-related macular degeneration.

The active ingredient of the medicine of the present invention (a polypeptide which inhibits binding between HB-EGF and an EGF receptor) specifically bounds with HB-EGF, and is advantageous in that it has potential effect on the patient to whom anti-VEGF antibody alone is not effective in intraocular angiogenesis. In addition, CRM-197 is a highly safe medicine which is already in the clinical trial stage.

The dose (usage) of the medicine of the present invention may be established as appropriate in consideration of the disease state, age, and body weight of the patient, and the form of the medicine. When the medicine is made into an injection for intravitreal injection, for example, the preparation containing 0.001 to 5% by weight of the active ingredient is administered once a day in an appropriate amount. When the medicine is made into an instillation, for example, the preparation containing 0.001 to 1% by weight of the active ingredient is administered to eyes once to several times a day, in an amount of one to several drops.

The target of the medicine of the present invention is not particularly limited, and includes human and mammal other than human (for example, pet animals, livestock, and experimental animals such as guinea pig, hamster, monkey, bovine, pig, goat, sheep, dog, cat, chicken, and partridge). The medicine of the present invention is preferably used for human.

As evident from the above statement, the present application also provides a prophylactic or therapeutic method including administering the medicine of the present invention to the patient with, for example, age-related macular degeneration, diabetic retinopathy, neovascular glaucoma, proliferative diabetic retinopathy, retinopathy of prematurity, exudative age-related macular degeneration, neovascular glaucoma, occlusion of retinal vein, retinal artery obstruction, pterygium, rubeosis, or corneal neovascularization in a therapeutically effective amount.

### EXAMPLES

### 1. Method

### 1-1. Laser-Induced Choroidal Neovascularization in Mouse

### 1-1-1. Mouse Model of Laser-Induced Choroidal Neovascularization

MYDRIN (registered trademark) P ophthalmic solution was dropped into the right eye of a mouse for causing mydriasis. A ten-fold dilution of a mixed anesthetic solution containing a 7:1 ratio of ketamine and xylazine with a normal saline solution (10 mL/kg) was administered into a femoral muscle. Thereafter, HYALEIN (registered trademark) ophthalmic solution 0.1% was dropped into the eyes for prevention of drying of the eyeballs. The eyeground was looked into while a cover glass was put to the right eye, and six points on the periphery of the optic disk were irradiated with laser at regular intervals (wavelength: 647 nm, spot size: 50 μm, irradiation time: 100 msec, laser output: 120 mW) using a laser beam coagulation apparatus (MC500; NIDEK CO., LTD, Aichi, Japan).

### 1-1-2. Making of Sample

Fourteen days after the laser irradiation, the mouse was anesthetized with a ten-fold dilution of a mixed anesthetic solution containing a 7:1 ratio of ketamine and xylazine with a normal saline solution (10 mL/kg), and 0.5 mL of fluorescein isothiocyanate dextran (FITC-dextran; 20 mg/mL ,Sigma-Aldrich) was administered into the tail vein of the mouse. The mouse was euthanized by cervical dislocation, and the eyeballs were extirpated. The extirpated eyeballs were fixed in a 4% paraformaldehyde phosphate buffer for 12 hours. Thereafter, the cornea and lens were excised under a microscope, and the remaining vitreous artery was removed by forceps. Furthermore, the retina was removed, the chorioid was notched 8 points, embedded with Fluoromount in a flat state, and thus made into a chorioid flat mount preparation.

### 1-1-3. Photographing and Quantitative Analysis Using Image Analysis Software

The chorioid flat mount was photographed using a confocal laser scanning microscope (FLUOVIEW FV10i; Olympus, Tokyo, Japan). By using the photographed image, the CNV was encircled using an analysis software OLYMPUS FLUOVIEW FV1000, and the area was recorded as CNV area (μm²).

### 1-2. Histological Study

### 1-2-1. Making of Mouse Model with Oxygen-Induced Retinal Angiogenesis

An oxygen-induced retinal angiogenesis model using newborn mice was made in accordance with the method of Smith et al. (Smith, L. E., Wesolowski, E., McLellan, A., Kostyk, S. K., D'Amato, R., Sullivan, R., and D'Amore, P. A. (1994) Oxygen-induced retinopathy in the mouse. Invest Ophthalmol Vis Sci 35, 101-111). The newborn mouse was bred from postnatal day 7 (P7) to P12 together with its parent mouse in a cage with a high oxygen concentration (75% O₂) regulated by an oxygen controller (PRO-OX 110; Reming Bioinstrumensts Co, Redfield, N.Y., USA). The oxygen concentration in the cage was measured twice a day using an oxygen controller. The newborn mouse was returned to the atmospheric pressure (21% O₂) on P12.

### 1-2-2. Making of Preparation of Mouse Model with Oxygen-Induced Retinal Angiogenesis

The mouse in the evaluation stage (P17) was deeply anesthetized by intraperitoneal administration of pentobarbital (20 mg/kg), and FITC-dextran with a molecular weight 2×10⁶, which is a fluorescent dye, was generally perfused from the left heart chamber at a rate of 20 mg/animal. After the perfusion, the eyeballs were extirpated, and fixed for 6 to 24 hours in a 4% paraformaldehyde phosphate buffer. The cornea and lenses were removed from the fixed eyeballs under a microscope, and the remaining vitreous artery was removed by forceps. Furthermore, the retina was peeled off, and embedded with Fluoromount (Diagnostic BioSystems, Pleasanton, Calif., USA) in a flat state, and thus making a retinal flat mount preparation.

### 1-2-3. Photographing of Retinal Blood Vessel Image and Quantitative Analysis Using Image Analysis Software

The retinal flat mount preparation was photographed under a fluorescent microscope (BX50, Olympus) using a high-sensitivity cooling CCD camera (DP3OBW, Olympus) through Metamorph (Universal Imaging Corp., Downingtown, Pa., USA) on an XY motor-operated stage (Sigma Koki Co., Ltd., Tokyo, Japan). The overview of the retina was made from 12 images. These images were continuously photographed from the surface layer to the lower layer of the retinal blood vessels at intervals of 14.2 μm.

The retinal blood vessels were quantified using Angiogenesis Tube Formation module in Metamorph. The setting items of this software were composed of the minimum blood vessel thickness, maximum blood vessel thickness, and luminance difference. The minimum blood vessel thickness was 1 μm. The maximum blood vessel thickness was measured three times at the most enlarged artery or vein, and the mean was used. The luminance difference was calculated by subtracting the background value from the luminance of the microvessel in the preparation. The background value is the mean of the three points, and the luminance value of the microvessel is the mean of five points, from which the maximum and minimum luminance values were excluded. The abnormal retinal blood vessels were compared between the HB-EGF-deficient and wild type mice on day 17 after birth, using the number of abnormal blood vessels and the area of abnormal blood vessels, which were obtained by Angiogenesis Tube Formation module, as the parameters. The abnormal blood vessels shows the number of clots thicker than the maximum blood vessel thickness, and the area of abnormal blood vessels shows the area of the clots.

### 1-3. Western Blot Analysis

### 1-3-1. Protein Extraction

Mouse eyeballs were extirpated, and the retinal pigment epithelium (RPE) -chorioid complex was isolated. The isolated tissues were placed in a microtube, and quickly frozen in liquid nitrogen. The sample was stored at −80° C. until protein extraction. The protein extract was mixtures of RIPA buffer with protease inhibiter cocktail, phosphatase inhibiter cocktail II, or III at the ratio of 100:1. 100 μL of a protein extract was added, and the microtube was homogenized for 1 minute in ice using a homogenizer (Psycotron, Microtec Co., Chiba, Japan). Thereafter, the components were allowed to react for 20 minutes in ice, and centrifuged at 10,000×g, 4° C., for 20 minutes. The centrifuged supernatant was collected, and used as a protein extract.

### 1-3-2. Protein Quantification and Protein Concentration Adjustment

Protein was quantified by using BCA Protein Assay kit. The standard was albumin standard at concentrations of 0 to 2,000 μg/mL, and the diluent was RIPA buffer containing no cocktail. The samples were diluted 10 folds with the diluent. After adding the Working reagent, the components were allowed to act for 30 minutes in a water bath 37° C., and then the absorbance at 532 nm was measured. The protein concentration was adjusted to 20 μg/mL using RIPA buffer, and a 1/4 amount the sample buffer (containing 20% 2-mercaptoethanol) was added to make the concentration 5 μg/mL. The prepared sample was stored at −80° C. until electrophoresis.

### 1-3-3. Electrophoresis and Transfer

The sample was taken out from −80° C., and returned to room temperature. Thereafter, the sample was boiled in boiling water at 100° C. for 5 minutes, and centrifuged at room temperature for 5 minutes at 10,000 rpm. SDS polyacrylamide gel (SuperSep 10%) was mounted on an electrophoresis apparatus, and a running buffer was placed in the vessel, and the electrophoresis apparatus equipped with gel was immersed. A running buffer was also placed in the electrophoresis apparatus. For one well, 5 μL of the molecular weight marker and 10 μL of the sample were placed. After adding the sample, electrophoresis was carried out at 20 mA for 90 minutes for one gel. After the electrophoresis, the gel was immersed in a cathode buffer (25 mM tris, 40 mM 6-amino-n-caproic acid, 20% methanol) for 15 minutes. The transfer membrane (Immobiron P) (Millipore, Billerica, Mass., USA) was immersed in methanol for 30 seconds, and immersed in ultrapure water for 15 minutes. Thereafter, the membrane was immersed in anode buffer 2 (25 mM tris, 20% methanol) for 15 minutes or more. From the anode side to the cathode side, filter paper immersed in anode buffer 1 (0.3 M tris, 20% methanol), filter paper immersed in anode buffer 2, transfer membrane, gel, and filter paper immersed in cathode buffer were tied up, and transferred for 45 minutes at 0.8 mA/cm².

### 1-3-4. Immunostaining

After the transfer, the transfer membrane was blocked by Block One-P for 30 minutes. Thereafter, the membrane was washed with Tris-buffer saline containing 0.05% tween (T-TBS), the first antibody was diluted with Can get signal solution 1, and allowed to react overnight at 4° C. After washing with T-TBS, the secondary antibody was diluted with Can get signal solution 2 at room temperature for 1 hour. After washing with T-TBS, the object was immersed in SuperSignal West Femto Maximum Sensitivity Substrate for 5 minutes. Thereafter, detection was carried out using Luminescent image analyzer LAS-4000 UV mini (Fujifilm, Tokyo, Japan). The primary antibodies were 1,000-fold dilutions of anti-HB-EGF antibody, anti-VEGF antibody, and anti-actin antibody, and the secondary antibodies were 2,000-fold dilutions of Horseradish peroxidase (HRP)-bound goat, rabbit, and mouse antibodies (Thermo Scientific).

### 1-3-5. Analysis of Protein Expression

The expression of protein was analyzed using Multi Gauge Ver 3.0 (Fujifilm) The band intensities were converted into numbers using Multi Gauge, and each value was calculated.

### 1-4. Immune Tissue Staining

Immediately after laser irradiation, using the sample on day 1, 3, 5, and 7 days, the chorioid was notched 8 points in the same manner as in 1-1-2, and then blocked at room temperature for 1 hour using 10% normal goat serum and 0.3% Triton X-100. After blocking, the sample was allowed to react overnight at 4° C. using a primary antibody. Thereafter, the sample was allowed to react for 1 hour using a secondary antibody with shielding light, and embedded with Fluoromount™ in a flat state. The primary antibodies were anti-HB-EGF antibody (1:100) and anti-VEGF antibody (1:100). The secondary antibodies were Alexa-633 conjugated goat anti-rabbit IgG (1:1,000) and Alexa-546 conjugated goat anti-rat IgG (1:1,000).

### 1-5. Study Using Human Retinal Microvascular Endothelial Cells

### 1-5-1. Cell Culture

Human retinal microvascular endothelial cells (HRMEC, DS Pharma Biomedical, Osaka, Japan) were cultured in a 10% FBS-containing CS-C medium [medium containing 10% FBS and Cell Boost] at 37° C., 5% CO₂. The medium was replaced 3 days after, and passage was carried out further 3 days after. The third to ninth passage cells were used in the experiment. The culture equipment was used after immersing the equipment surface in the cell adhesion factor, and thoroughly conforming the surface to the factor.

### 1-5-2. Proliferation Test

HRMEC was seeded on a 96-well plate at a density of 2,000 cell/well, cultured for 24 hours, at 37° C., 5% CO₂, and then the medium was replaced with 10% FBS CS-C medium (medium containing no Cell Boost), and cultured for 24 hours at 37° C., 5% CO₂. HB-EGF and VEGF or HB-EGF and VEGF were added simultaneously to HRMEC to make the final concentration 1 to 10 ng/mL and 10 ng/mL, respectively, and CRM-197 was added 1 hour before the addition of HB-EGF and VEGF to make the final concentration 10 μg/mL. The cells were further cultured for 48 hours in a 10% FBS CS-C medium, and then CCK-8 was added to each well, and incubated for 3 hours at 37° C., 5% CO₂. CCK-8 contains a tetrazolium salt [2-(2-methoxy-4-nitrophenyl)-3-(4-nitrophenyl)-2H tetrazolium monosodium salt: WST-8]. WST-8 (colorless) is reduced by dehydrogenase in living cells in the presence of an electron mediator 1-methoxy-5-methylphenazium methyl sulfate (1-methoxy PMS), and forms water-soluble formazan (orange). The formazan absorbance 492 nm (control wavelength 660 nm) was directly measured, thereby measuring the living cells.

### 1-5-3. Migration Test

A 12-well plate was coated with 0.03% collagen, and HRMEC was seeded at adensity of 40,000 cell/well. The cells were cultured for 24 hours at 37° C., 5% CO₂.

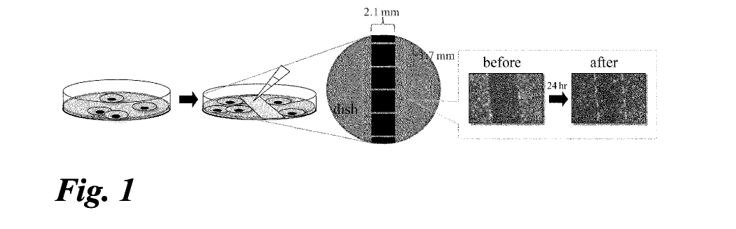

Thereafter, the cells were transferred to 1% FBS CS-C medium (a medium containing no Cell boost), and incubated for 6 hours. Thereafter, the cells on the center line of the well were peeled off using a 1000 μl-chip (TR-222-C, Axcygen Scientific, Central Avenue, CA, USA), washed with PBS, and the medium was replaced. Immediately after that, four points on each well were phogotraphed (before migration) using a high-sensitivity cooling charge coupled device (CCD) camera (DP30BW, OLYMPUS, Tokyo, Japan) (3.6 mm²/1 point). HB-EGF and VEGF or HB-EGF and VEGF were added simultaneously to make the final concentrations at 0.1 to 10 ng/mL and 10 ng/mL, respectively. CRM-197 was added one hour before the addition of HB-EGF and VEGF to make the final concentration 10 μg/mL. After incubating for 24 hours, at 37° C., 5% CO₂, each well was photographed in the same manner as described above. The number of the cells which moved to the place detached from the place before migration was counted, and the mean of four points for each well was calculated (FIG. 1).

### 1-6. Statistical Analysis

The statistical analysis used Student's t-test, Dunnett's test, or Tukey's test. The experimental result was expressed in the mean±S.E.M, and the risk rate of 5% or less was rated as significant.

### 2. Result

### 2-1. Study of Influence in Laser-Induced Choroidal Neovascularization Model of HB-EGF Deficient Mouse

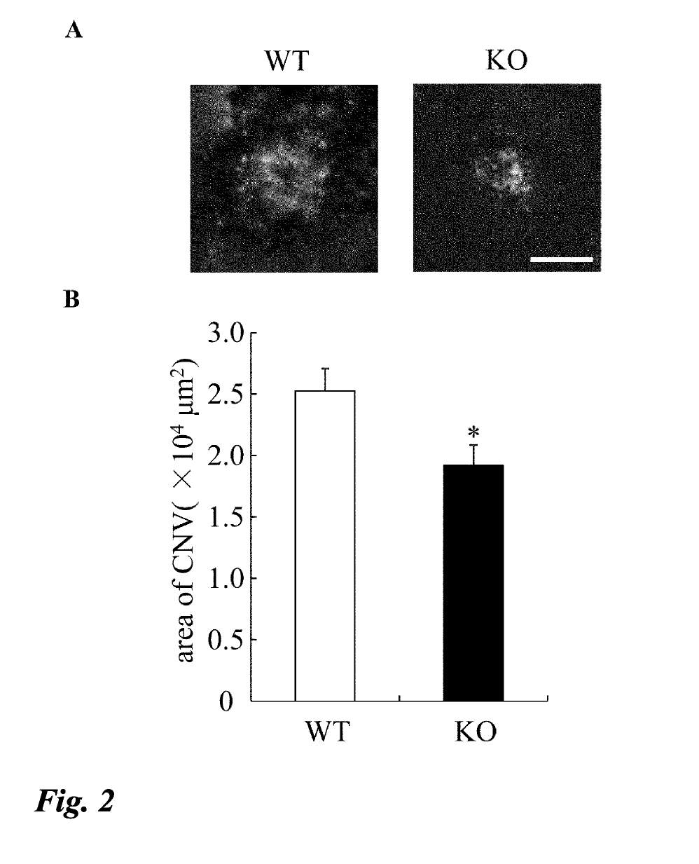

The CNV expression area in the HB-EGF deficient mouse was 24% lower than that in the wild type mouse (FIG. 2).

### 2-2. Study of Influence in Oxygen-Induced Model in HB-EGF Deficient Mouse

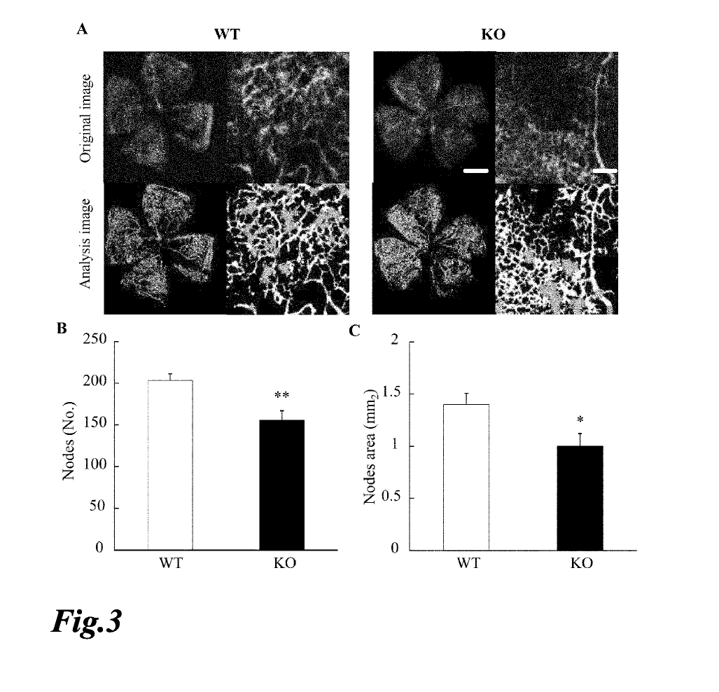

The number of abnormal blood vessels was 23% lower, and the area of abnormal blood vessels was 28% lower in the HB-EGF deficient mouse than those in the wild type mouse (FIG. 3).

### 2-3. Change of HB-EGF and VEGF Expression Amounts in Mouse Laser-Induced Choroidal Neovascularization Model

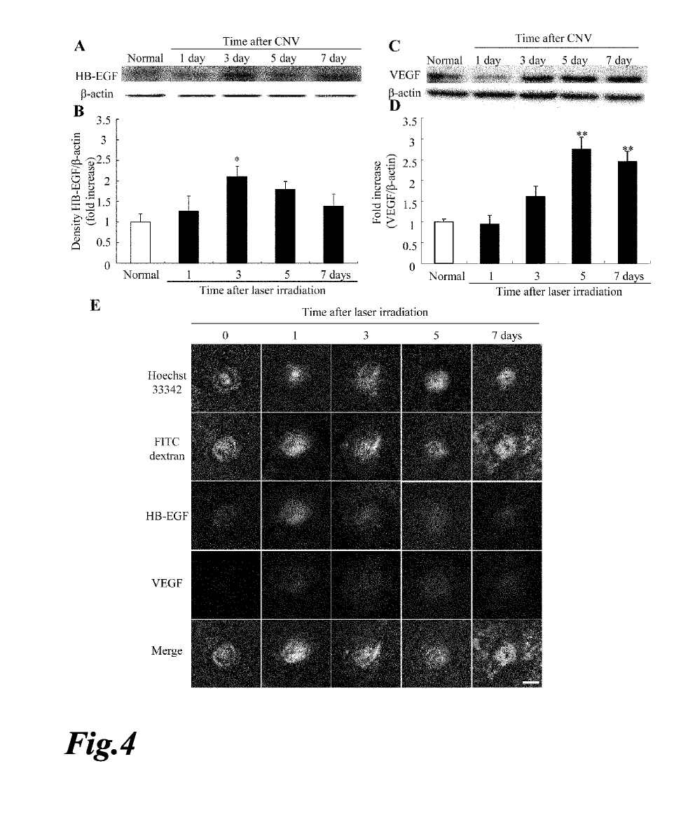

The changes of HB-EGF and VEGF in the laser-induced choroidal neovascularization model were studied. Expression of HB-EGF significantly increaseed on day 3 after laser irradiation. On the other hand, expression of VEGF significantly increase on days 5 and 7 after laser irradiation (FIG. 4). Furthermore, HB-EGF and VEGF colocalized in the newborn blood vessel regions after laser irradiation (FIG. 4).

### 2-4. Action of HB-EGF on Proliferation of HRMEC

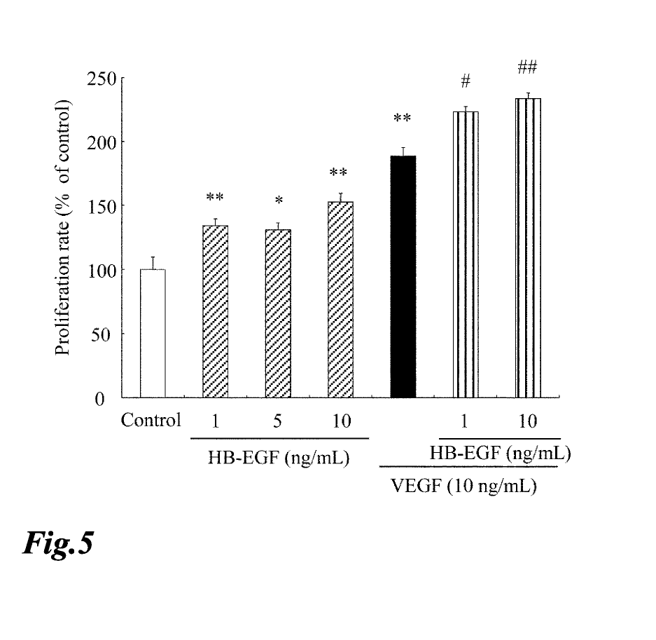

In order to study the influence of HB-EGF on the retinal vascular endothelial cells, human recombinant HB-EGF was added to HRMEC, and the cell proliferation capacity was evaluated. HB-EGF (1 to 10 ng/mL) accelerated the proliferation of HRMEC in a concentration-dependent manner, and was significant at the concentrations of 1, 5, and 10 ng/mL (FIG. 5). Furthermore, the simultaneous addition of HB-EGF and VEGF further accelerated the proliferation of HRMEC in a concentration-dependent manner, and was significant at the concentrations of 1 and 10 ng/mL (FIG. 5).

### 2-5. Action of HB-EGF on Migration of HRMEC

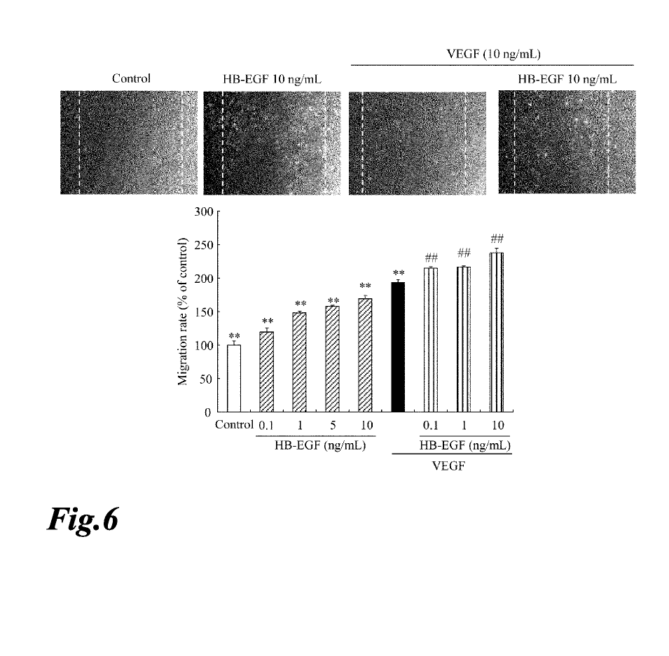

The migration capacity of HRMEC was evaluated using wound-healing assay (Nakamura S, Hayashi K, Takizawa H, Murase T, Tsuruma K, Shimazawa M, Kakuta H, Nagasawa H, Hara H. (2011) An arylidene-thiazolidinedione derivative, GPU-4, without PPARy activation, reduces retinal neovascularization. Curr Neurovasc Res. 8 (1), 25-34.). Human recombinant HB-EGF significantly accelerated migration of HRMEC in a concentration-dependent manner (FIG. 6). The addition of HB-EGF (0.1, 1, 5, and 10 ng/mL) accelerated the migration about 1.3 to 1.8 times in comparison with the control group. Furthermore, the simultaneous addition of HB-EGF and VEGF significantly accelerated the cell migration in comparison with the VEGF alone group (FIG. 6).

### 2-6. Action of CRM-197 on HRMEC Proliferation

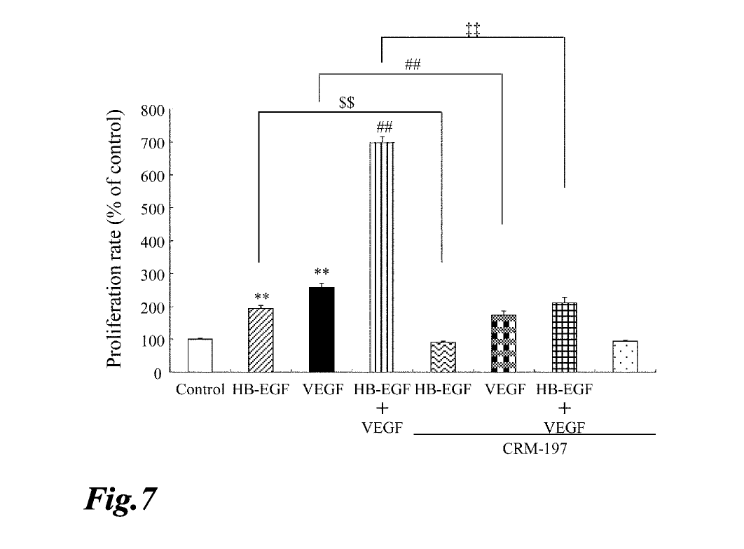

In order to study the influence of HB-EGF on the retinal vascular endothelial cells, CRM-197 as an inhibitor of HB-EGF was added to HRMEC, and the action of CRM-197 in the proliferation of HB-EGF-induced cells was evaluated. CRM-197 (10 μg/mL) showed inhibitory action on the proliferation of HB-EGF-induced and VEGF-induced HRMEC (FIG. 7).

### 2-7. Action of CRM-197 on HRMEC Migration

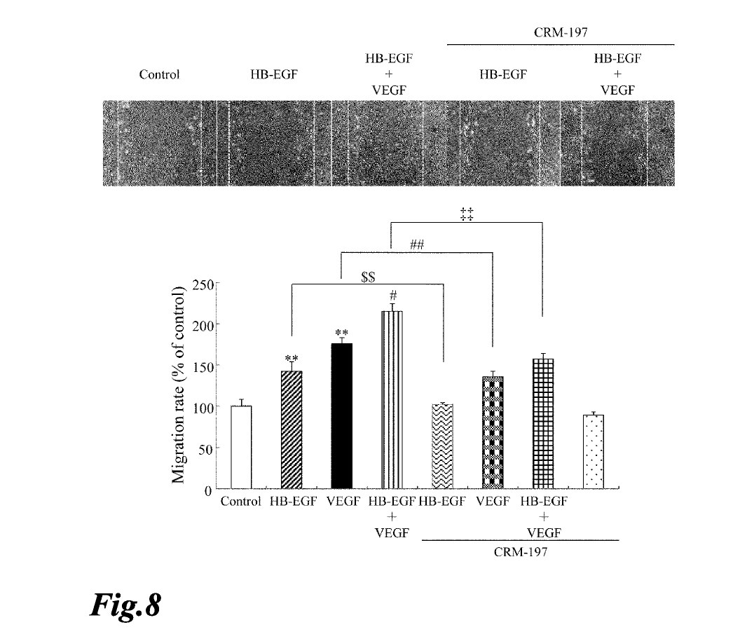

In order to study the influence of HB-EGF on the retinal vascular endothelial cells, CRM-197 as an inhibitor of HB-EGF was added to HRMEC, and the action of CRM-197 in the migration of HB-EGF-induced cells was evaluated. CRM-197 (10 μg/mL) significantly inhibited the migration of the HB-EGF-induced and VEGF-induced HRMEC. Furthermore, CRM-197 (10 μg/mL) significantly inhibited cell migration also in the group simultaneously treated with HB-EGF and VEGF (FIG. 8).

### 3. Summary

It was proved that HB-EGF is involved with intraocular angiogenesis, and that intraocular angiogenesis can be an effective treatment target of HB-EGF. It was also proved that CRM-197 effectively suppresses the action of HB-EGF. CRM-197 can be regarded as a prophylactic or therapeutic medicine for ophthalmologic diseases accompanied by intraocular angiogenesis.

The present invention is not limited only to the description of the above embodiments. A variety of modifications which are within the scopes of the following claims and which are achieved easily by a person skilled in the art are included in the present invention.

What is claimed is:

1. A prophylactic or therapeutic method for an ophthalmologic disease accompanied by intraocular angiogenesis, comprising a step of administering a polypeptide to a patient with ophthalmologic diseases accompanied by intraocular angiogenesis in a therapeutically effective amount, wherein the polypeptide is a variant of diphtheria toxin and shows activity of inhibiting binding between HB-EGF and an EGF receptor.

2. The method of claim 1, wherein the polypeptide is CRM-197.

3. The method of claim 1, wherein the ophthalmologic disease is age-related macular degeneration, diabetic retinopathy, neovascular glaucoma, proliferative diabetic retinopathy, retinopathy of prematurity, exudative age-related macular degeneration, neovascular glaucoma, occlusion of retinal vein, retinal artery obstruction, pterygium, rubeosis, or corneal neovascularization.

4. The method of claim 2, wherein the ophthalmologic disease is diabetic retinopathy, proliferative diabetic retinopathy, or exudative age-related macular degeneration.

5. An intraocular angiogenesis inhibitor including a polypeptide which is a variant of diphtheria toxin, and shows activity inhibiting binding between HB-EGF and an EGF receptor.

6. The intraocular angiogenesis inhibitor of claim 5, wherein the polypeptide is CRM-197.

7. A prophylactic or therapeutic medicine for an ophthalmologic disease accompanied by intraocular angiogenesis, including the intraocular angiogenesis inhibitor of claim 5 as an active ingredient.

8. The prophylactic or therapeutic medicine of claim 7, wherein the ophthalmologic disease is age-related macular degeneration, diabetic retinopathy, neovascular glaucoma, proliferative diabetic retinopathy, retinopathy of prematurity, exudative age-related macular degeneration, neovascular glaucoma, occlusion of retinal vein, retinal artery obstruction, pterygium, rubeosis, or corneal neovascularization.
